(19) United States
(12) Patent Application Publication
(10) Pub. No.: US 2007/0117132 A1
(43) Pub. Date: May 24, 2007

(54) System and method for analyzing dependence between gene expressions

(76) Inventors: Andrei Yakovlev, Pittsford, NY (US); Lev Klebanov, Prague (CZ); Craig Jordan, Rochester, NY (US)

Correspondence Address: BLANK ROME LLP, 600 NEW HAMPSHIRE AVENUE, N.W., WASHINGTON, DC, 20037, US

(21) Appl. No.: 11/593,635

(22) Filed: Nov. 7, 2006

Related U.S. Application Data
(60) Provisional application No. 60/734,271, filed on Nov. 8, 2005.

(51) Int. Cl. C12Q 1/68; G06F 19/00

(52) U.S. Cl. 435/6; 702/20

(57) ABSTRACT

An amplification-like, unidirectional dependence between expression levels in gene pairs is identified through statistical analysis including a determination of whether there is a specified level of independence (or non-correlation) between X and Z, where X is a random variable representing the expression level of a first gene, Y is a random variable representing the expression level of a second gene, and Y=XZ where Z≧1. The disclosed process provides an ability to identify genes that act as “amplifiers,” thereby facilitating appropriate priority assignment to candidate genes for further evaluation and experimentation.

### REFERENCE TO RELATED APPLICATION

The present application claims the benefit of U.S. Provisional Patent Application No. 60/734,271, filed Nov. 8, 2005, whose disclosure is hereby incorporated by reference in its entirety into the present disclosure.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant GM075299 awarded by NIH/NIGMS.

### FIELD OF THE INVENTION

This invention relates generally to the field of identifying relationships between genes using statistical analysis.

### DESCRIPTION OF RELATED ART

It has become common practice to use microarray technology for finding “interesting” genes by comparing two or more different phenotypes. Modern methods of microarray data analysis typically employ two-sample statistical tests combined with multiple testing procedures to control a Type 1 error rate.

Such methods generally select those genes that display the most pronounced differential expression. Once the list of genes showing statistically significant differential expression has been generated, these genes are often ranked using purely statistical criteria and this ranking is intended to reflect their relative importance. Typically, a certain number of genes with the smallest p-values is finally selected from the list of all “significant” ones. While most biologists recognize that the magnitude of differential expression does not necessarily indicate biological significance, this approach is the one conventionally used for initial prioritizing of candidate genes.

From a biological perspective, the above-described paradigm falls far short of being a perfectly valid approach. Even a very small change in expression of a particular gene may have dramatic physiological consequences if the protein encoded by this gene plays a catalytic role in a specific cell function.

The inventors have determined that downstream genes may amplify the signal produced by this truly interesting gene, thereby increasing their chance to be selected by formal statistical methods. For a regulatory gene, however, the inventors have determined that the chance of being selected by such methods often diminishes as one keeps hunting for downstream genes which tend to show much bigger changes in their expression. As a result, the final list of candidates may be enriched with many effector genes that do little to elucidate more fundamental mechanisms of biological processes.

Recent years have seen a growing interest in correlations between gene expression levels in statistical methodologies for microarray analysis. For example, see the following articles: Xiao Y., Frisina R., Gordon A., Klebanov L., Yakovlev A. (2004) “Multivariate search for differentially expressed gene combinations,” BMC Bioinformatics 5: # 164; Dettling M., Gabrielson E., Parmigiani G. (2005) “Searching for differentially expressed gene combinations,” http://www.bepress.com/jhubiostat/paper77; Qiu X., and Brooks A. I., Klebanov L., Yakovlev A. (2005) “The effects of normalization on the correlation structure of microarray data,” BMC Bioinformatics 6: # 120.

### SUMMARY OF THE INVENTION

The inventors have determined that there is a need for an improved method of analyzing dependence between gene expressions. It is therefore an object of the invention to provide one.

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In an exemplary embodiment, a statistical process is used to determine whether there is a specified level of independence (or non-correlation) between X and Y/X, where X is a random variable representing the expression level of a first gene, Y is a random variable representing the expression level of a second gene, and Y=X Z where Z≧1. The disclosed process enables identification of genes that act as “amplifiers,” and can be used to assign appropriate priorities to candidate genes for further evaluation and experimentation.

Additional features and advantages of the invention will be set forth in the description which follows, and in part will be apparent from the description, or may be learned by practice of the invention. The advantages of the invention will be realized and attained by the structure particularly pointed out in the written description and claims hereof as well as the appended drawings.

The following paper is hereby incorporated by reference in its entirety into the present disclosure: Klebanov, L., Jordan, C., and Yakovlev, A., “A new type of stochastic dependence revealed in gene expression data,” Statistical Applications in Genetics and Molecular Biology, 2006, vol. 5, Article 7.

### BRIEF DESCRIPTION OF THE DRAWINGS

The accompanying drawings, which are incorporated herein and form a part of the specification, illustrate various exemplary embodiments of the present invention and, together with the description, further serve to explain various principles and to enable a person skilled in the pertinent art to make and use the invention.

FIG. 1 is a flow chart showing a process for categorizing genes as Type A or Type B according to an exemplary embodiment.

FIG. 2 is a diagram illustrating the stability of the disclosed inventive procedure for gene selection for 500 randomly selected genes.

FIG. 3 is a correlation diagram illustrating the difference between the A and B types of gene pairs in an exemplary analysis.

FIG. 4 is a block schematic diagram showing an example of a computer system that can be used to implement some embodiments.

### DETAILED DESCRIPTION OF THE PREFERRED EMBODIMENTS

The present invention will now be described with reference to the accompanying drawings. In the drawings, some like reference numbers indicate identical or functionally similar elements. Additionally, the left-most digit(s) of most reference numbers may identify the drawing in which the reference numbers first appear.

The present invention will now be explained in terms of exemplary embodiments. This specification discloses one or more embodiments that incorporate the features of this invention. The disclosure herein will provide examples of embodiments, including examples of data analysis from which those skilled in the art will appreciate various novel approaches and features developed by the inventors. These various novel approaches and features, as they may appear herein, may be used individually, or in combination with each other as desired.

In particular, the embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, persons skilled in the art may effect such feature, structure, or characteristic in connection with other embodiments whether or not explicitly described.

Embodiments of the invention may be implemented in hardware, firmware, software, or any combination thereof, or may be implemented without automated computing equipment. Embodiments of the invention may also be implemented as instructions stored on a machine-readable medium, which may be read and executed by one or more processors. A machine-readable medium may include any mechanism for storing or transmitting information in a form readable by a machine (e.g. a computing device). For example, a machine-readable medium may include read only memory (ROM); random access memory (RAM); hardware memory in PDAs, mobile telephones, and other portable devices; magnetic disk storage media; optical storage media; flash memory devices; electrical, optical, acoustical, or other forms of propagated signals (e.g. carrier waves, infrared signals, digital signals, analog signals, etc.), and others. Further, firmware, software, routines, instructions, may be described herein as performing certain actions. However, it should be appreciated that such descriptions are merely for convenience and that such actions in fact result from computing devices, processors, controllers or other devices executing the firmware, software, routines, instructions, etc.

The present invention arises from a discovery by the inventors that many genes have a very special type of relationship with each other that has not been identified in prior research.

This newly identified relationship will be described as either “Type A” or “Type B” dependence and the corresponding gene pair will be referenced as a “Type A” or “Type B” pair. The categorization of gene pairs as Type A or Type B is useful, for example, in screening genes to determine priorities for further research. As will be seen, Type B genes are biologically more likely to be good candidates for evaluation.

For purposes of explaining theories that may support exemplary embodiments of the invention, “Type A” dependence will be defined as follows. Let X and Y be random variables (RVs) representing the expression levels of genes gₓ and g_{y}. A pair of genes (gₓg_{y}) is said to be of Type A if X and Y satisfy the condition:
Y=XZ (1)

where Z is a positive RV which is stochastically independent of X.

Those gene pairs that do not display the Type A dependence are defined as “Type B” pairs.

It follows from the above definitions that the RVs X and Y/X are independent in a Type A gene pair. At the same time, the RVs X and Y are stochastically dependent so that the regular correlation between them can be quite strong. Note that the roles of gₓ and g_{y} in such a pair are not symmetrical because, assuming Equation (1) is true, the RVs Y and 1/Z in the relationship X=Y/Z are no longer independent. The dependence (1) is thus unidirectional. For purposes of describing exemplary embodiments of the invention, gₓ will be described herein as the “driver” (DR) and g_{y} will be described as the “amplifier”(AM).

Equation (1) implies an amplification-type relationship between X and Y that can be interpreted as follows. Suppose gene gₓ produces transcripts with intensity X, then gene g_{y} produces Z transcripts per one transcript produced by gene gₓ. In other words, gene g_{y} amplifies gene gₓ at the transcription level. This kind of amplification is understood in a broad sense with the amplification being positive if the mean value of Z is greater than 1 and negative otherwise. Gene g_{y} may play the role of a driver in some other gene pair of Type A. Likewise, gene gₓ may be an amplifier in another Type A pair. Both genes may also form Type B relationships with other genes.

Stochastic dependence (as it relates to Equation 1) does not necessarily imply a direct causal relationship between genes gₓ and g_{y}; it can also be thought of as an association between them arising from the assignment of operations to different genes by a molecular mechanism residing outside the pair. In other words, there are no grounds to interpret a chain of Type A pairs in terms of biochemical pathways; they are in a sense a “supply chain.” Even with this cautious interpretation, all genes in Type A pairs may not play any major regulatory role in a given phenotype because a unidirectional dependence of this type cannot accommodate a feedback circuit. Such pairs still may be of great biological interest but in a domain which is different from what biologists typically seek to obtain from gene expression data. In a preferred embodiment, Type B pairs are considered as promising candidates for further exploration.

As will be seen, there is evidence that the reported type of dependence is real and not just a random pattern seen in a particular data set. In doing so, the inventors have estimated the abundance of Type A pairs, assessed stability of their selection, and provided specific examples of their patterns in microarray data.

The procedure described herein is designed to reject the null hypothesis H₀: the gene pair under consideration is a Type A pair. Therefore, it is necessary to estimate the expected proportion π_{A} of true null hypotheses in the data at hand. In a preferred approach, no multiple testing adjustment is employed. It is desirable to obtain a lower bound for this proportion; the more hypotheses rejected the lower the estimate. Also, the preferred procedure for rejecting H₀ is anti-conservative by design, as it allows for the presence of a multiplicative random noise in the data. This allows the approach to be as conservative as possible when estimating π_{A}.

In accordance with the method disclosed in Storey, J. D. & Tibshirani, R. “Statistical significance for genomewide studies,” Proc. Natl. Acad. Sci. USA 100, 9440-9445 (2003), π_{A} can be estimated by a limiting value of

$${\hat{π}}_{A} ( λ ) = \frac{\# \{ p 〉 λ ; i = 1 , … , m \}}{m ( 1 - λ )} \qquad ( 2 )$$

as the parameter λ tends to 1. In Equation (2), pᵢ(i=1, . . . , m) are the observed p-values calculated from quantiles of the test described herein, and m is the total number of the hypotheses tested.

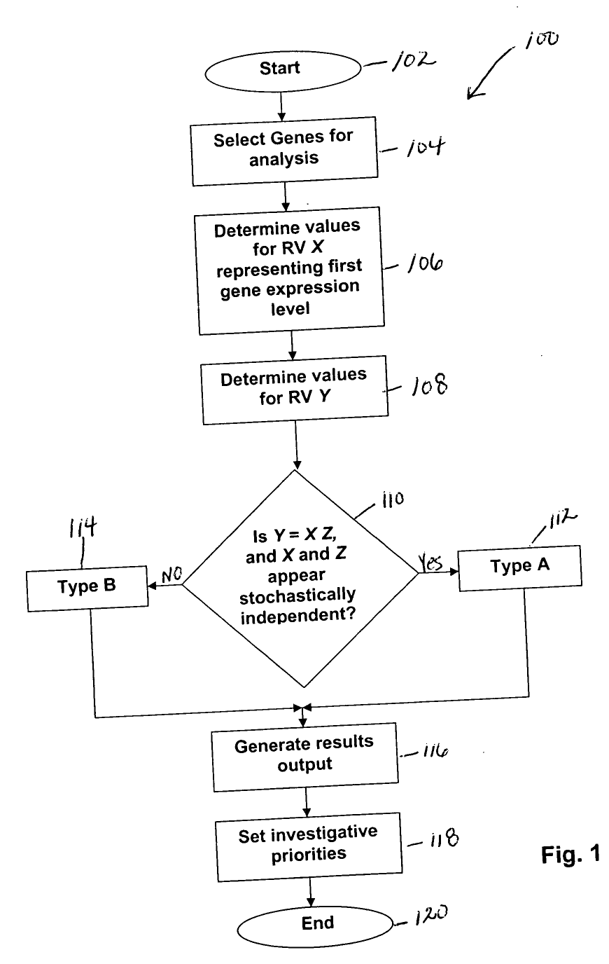

FIG. 1 is a flow chart showing a generalized process and method 100 for identifying Type A and Type B gene pairs. The method begins at step 102 and proceeds to step 104 where a pair of genes to be analyzed is selected. Two specific genes may be selected or a large number of genes may be paired and their paired relationships may be analyzed either sequentially or in parallel.

In step 106, values are obtained for the random variable X, representing the expression level of the first gene in the pair. In step 108, similarly, values are obtained for the random variable Y, representing the expression level of the second gene in the pair.

In step 110, the stochastic independence of X and Z is estimated and a Type A or B relationship is predicted according to the definitions noted above. This step can be accomplished either through direct analysis to determine whether these variables are substantially independent, or preferably may be accomplished through a correlation analysis and with approximation in other regards such that a generally reliable guide as to whether the genes have a Type A or Type B relationship can be obtained. Any of the equations, methods, and approximations set forth in this disclosure can be applied to determine whether a gene has a type A or type B relationship with another gene. Further, those skilled in the art, having reviewed the present specification, will recognize that the exemplary equations and statistical approaches described herein can be modified to incorporate other known mathematical and scientific methods without departing from the spirit of the invention.

If the requirements for a Type A relationship are apparently met by the gene data, control passes to step 112 and then to step 116. If the requirements for a Type A relationship are not met, control passes to step 114 (Type B established) and then to step 116.

In step 116, a results output is generated to indicate, typically, at least those genes of Type B. Various information obtained during the analysis may be output as desired. For example, information identifying Type A genes, information describing statistical certainty of results, information identifying which genes are DR and which are AM in a pair, and other information may be output as desired.

In step 118, investigative priorities may be set taking into account a variety of research information, including for example one or more data from the output generated in step 116.

The example process concludes in step 120. Although omitted from the flow chart of FIG. 1 for clarity, it will be understood that the comparison process between two genes implemented in steps 104-116 may be repeated iteratively or otherwise, to identify genes that are drivers for identified driver genes.

It should also be noted that the embodiments are not limited to the steps disclosed herein. These steps can be modified as desired, augmented with other steps, and the order of the steps can be changed without departing from the spirit of the invention.

In another exemplary embodiment, an example analysis was performed using three subsets of data identified through the St. Jude Children's Research Hospital (SJCRH) Database on childhood leukemia (http://www.stjuderesearch.org/data/ALL1) were analyzed. The SJCRH Database contains gene expression data on 335 subjects, each represented by a separate array (Affymetrix, Santa Clara, Calif.) reporting measurements on the same set of m=12550 genes. In this example, the inventors selected the following three groups of patients: Group 1 was represented by 19 arrays obtained from patients with acute lymphoblastic leukemia characterized by a normal cytogenic phenotype NORMAL, Group 2 included 45 patients with T-cell acute lymphoblastic leukemia TALL, and Group 3 was represented by 88 patients with hyperdiploid (Hyperdip) acute lymphoblastic leukemia.

Since the available sample size was sufficient for the use of distribution-free methods, the inventors applied the Cramer-von Mises two-sample test with Bonferroni adjustment to compare Group 1 (NORMAL) and Group 2 (TALL) of the patients with childhood leukemia. This test resulted in 342 differentially expressed genes when controlling the familywise error rate at the 0.05 level.

In this example, simplified calculations are implemented to approximate a resolution of Equation (2) for a particular gene pair. A log-transformation of Equation (1) above provides:
y=x+z (3)

where x=log X, y=log Y, and z=log Z. Therefore, testing the hypothesis H₀, the gene pair under consideration is a Type A pair is equivalent to testing the hypothesis: x and z=y−x are stochastically independent. Testing of independence is generally more cumbersome and time-consuming than testing to determine an absence of correlation. Therefore, the approach of calculating a correlation level and making a decision based on that level is preferred in most cases. Sample analyses indicate that correlation level testing provides good agreement with the results of independence testing.

A statistical test for the hypothesis H′₀: ρ(x, z) =0, where ρ(x, z) is the correlation coefficient between x and z, is given by

$$ω = \frac{1}{2} log \frac{1 + r ( x , z )}{1 - r ( x , z )} \qquad ( 4 )$$

where r is the Pearson sample correlation coefficient. The statistic ω has an asymptotic normal distribution with mean 0 and variance 1/\sqrt{n−1}.

Thus, the hypothesis that the correlation coefficient between x and y−x is equal to zero can be tested to make a decision about whether or not a given pair is of Type A. However, the situation is more complicated if there is multiplicative-array-specific technological noise in the data because the correlation structure of vector (x, z) becomes non-identifiable. To surmount this difficulty, it is desirable for the test to be anti-conservative, thereby rejecting more true null hypotheses. In the presence of a multiplicative noise denoted by Θ, one can observe (θ+x, θ+y), where θ=log Θand Θ is a RV independent of the pair (X, Y). Consider the pair of RVs (x+θ, z), where z=(y+θ) (x+θ). It can be shown that:

$$ρ ( x , z ) = ρ ( x + θ , z ) {\{ 1 + \frac{{σ}^{2} ( θ )}{{σ}^{2} ( x )} \}}^{1 / 2} \qquad ( 5 )$$

Unfortunately, x and θ are not directly observable so that Equation (5) cannot be used directly. Since for any x, σ² (x+θ)=σ² (x)+σ²(θ)≧σ₂ (θ), therefore,

$${σ}^{2} ( θ ) \leq {σ}^{2} = {min}_{1 \leq i \leq m} {σ}_{i}^{2} ( x + θ ) \qquad ( 6 )$$

where σᵢ²(x+θ) is the variance of observed (noisy) expression of the i th gene and the minimum in Equation (6) is taken over all genes. Notice also that σ² (x)≧σ²(x +θ)−σ². Therefore, the coefficient ρ(x, z) satisfies the following inequality:

$$ρ ( x , z ) \leq ρ ( x + θ , z ) {\{ 1 + \frac{{σ}^{2}}{{σ}^{2} ( x + θ ) - {σ}^{2}} \}}^{1 / 2} \qquad ( 7 )$$

where all the characteristics involved can be estimated for all genes except the one for which the minimum in Equation (6) is attained. The latter gene is discarded.

By replacing all the parameters in the right-hand side of Equation (7) with their empirical estimators, there is obtained a test-statistic for rejecting H′₀. The asymptotic distribution of this statistic is also normal, but with a larger variance. Therefore, the test becomes even more anti-conservative when using percentiles of the distribution for the statistic ω given by Equation (4). Being anti-conservative by design, this test leads to a conservative estimate of the abundance of Type A gene pairs in the data at hand, which is consistent with the preferred goal of establishing the existence of Type A dependence beyond a reasonable doubt before indicating that a pair has a Type A relationship.

While the estimate of π_{A} resulting from Equation 2 (or its surrogates obtained by the correlation approximation method described above) can be highly variable if p-values are heavily correlated, its variance can be estimated by resampling. As an example, using three publicly available sets of data, the inventors studied the expression profiles of all pairs of genes formed from a total of 12550 genes (probe sets).

In an example variance analysis, the inventors randomly selected 1500 genes and applied the delete-d-jackknife method (with 200 subsamples) to the collection of arrays for Group 2(d=6) and Group 3(d=8) of patients with childhood leukemia. The above estimation procedure was applied to obtain the estimate \hat{π}_{A} from each subsample, and then the mean E(\hat{π}_{A}), median M(\hat{π}_{A}), and standard deviation S(\hat{π}_{A}) were computed from the 200 subsamples. The latter characteristic was estimated by a resampling counterpart of the jackknife sample standard deviation, as described in Shao, J. & Tu, D. “The Jackknife and Bootstrap,” Springer Series in Statistics, Springer, New York (1995). The resultant estimates were: E(\hat{π}_{A})=46%, M(\hat{π}_{A})=46%, S(\hat{π}_{A})=5% for Group 2, and E(\hat{π}_{A})=43%, M(\hat{π}_{A})=43%, and S(\hat{π}_{A})=6% for Group 3. When both datasets were pooled together, thereby increasing the power of this test for H₀, the estimates changed but slightly: E(\hat{π}_{A})=35%, M(\hat{π}_{A})=34%, S(\hat{π}_{A})=4.5%.

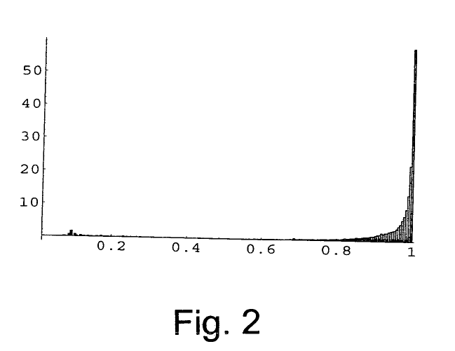

The stability of the proposed procedure for gene selection was also assessed by resampling. The results of this assessment for 500 randomly selected genes are shown in FIG. 2. As can be observed in FIG. 2, the selection stability is remarkably high, indicating a stable pattern in the data under study.

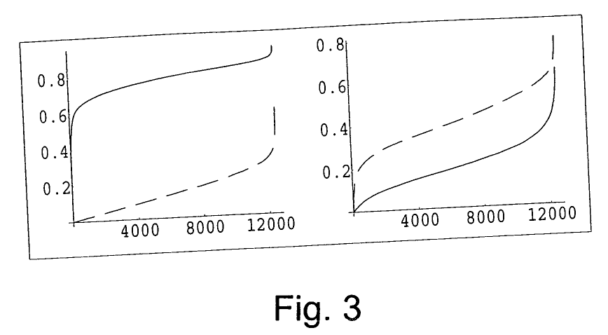

FIG. 3 shows a correlation diagram that illustrates an example of a difference between Type A and Type B gene pairs. To produce FIG. 3, Pearson correlation coefficients were computed for all pairs formed by a given gene gₓ. The solid line in FIG. 3 represents these coefficients in increasing order. In each pair (gₓ, g_{y}), correlations between X and Y/X and between Y and X/Y, respectively, are also computed and the minimum of the two values is selected. The resultant correlation coefficients for all pairs formed by the chosen gene are plotted in increasing order (dashed line).

FIG. 3 shows a reversal of the type of dependence for gene TCL1A when comparing TALL (the left panel) with NORMAL (the right panel). The x-axis in FIG. 3 shows the ordered pair number, while the y-axis is the correlation coefficient.

From the joint behavior of the two curves, it is clear that TCL1A tends to form Type A relationships with the overwhelming majority of genes in TALL. The pattern seen in TALL is reversed in the NORMAL data where TCL1A forms Type B relationships with the majority of genes. Therefore, the prevalence of type A over type B relationships and vice versa provides additional information on each gene. In an embodiment, the methodology thus described can be used to categorize a gene as Type A or Type B.

The inventors believe that the relationship DR→AM, e.g. amplification-like dependence, manifesting itself in type A gene pairs, is a stable mass phenomenon. Such pairs can form long chains, as will be described herein, that change their structure (membership) under different conditions. The information on Type A dependencies can be utilized for screening, e.g. to narrow the set of differentially expressed genes resulted from two-sample comparisons, enabling focusing of further research on more likely genes. In a comparison of two groups of patients with leukemia (designated Group 1 and Group 2), a total of 342 genes were declared differentially expressed.

In an example analysis, the inventors used 1000 cross-validations to select only stable (with 100% selection frequency) Type A pairs and to discard all genes that were classified as AMs by the method described herein. This procedure resulted in as few as 49 finally selected genes. The names of these genes are as follows: DUSP 1, PLCG2, CTSC, TCF3, PTPN18, TGFBR2, EST, ENG, NDUFB8, TNFAIP8, ENOSF1, TBXA2R, TP11, DNTT, GNPDA1, KIAA0063, PAPSS1, CD96, GALNAC4S-6ST, TSC22D3, BTG2, ETFB, SYK, GPX1, IGHD, PALM, MEF2C, IL27RA, CD47, GNAQ, CDK9, HDAC5, GP1, CAPN3, TCL1A, IL1B, CUL1, PFKL, ICAM3, HLA-G, FNBP1, BEXL1, B4GALT1, SCARB1, EST, KIAA0152, GNA11, HLX1, FYB, DHRS7, HLA-F, STAT5A, CD9, CD22 MAG, SLC2A5, EST.

Among the 49 genes selected, specific classes are preferentially enriched. For example, genes defined as transcription factors or adhesion molecules show increased frequencies between approximately 50% and 100%, respectively. Similarly, genes regulating various aspects of metabolism also increase 100% in the set of 49 genes.

Thus, the inventors conclude that this selection procedure is likely to yield information about the general nature of genes that mediate leukemia-specific processes. This approach may not select the genes with the largest differences in expression levels. Indeed, there were only 3 out of the 49 genes found among the 50 most differentially expressed genes with the smallest adjusted p-values. One of them (TCL1 A) was the 6th top gene in terms of differential expression while the other two (GALNAC4S-6ST and ENG) were the 40th and 42nd, respectively.

Thus, the inventors have identified a novel method of analyzing microarray gene expression data. The presence of the phenomenon identified by the inventors has been corroborated by similar analyses of several smaller data sets. The method used in this analysis is robust to random technological noise.

Those skilled in the art will recognize that specially designed experiments will demonstrate additional details of the DR-AM relationships between and the putative mechanism by which the cell distributes various tasks among genes. In particular, gene perturbation experiments conducted as a subsequent step to the analysis disclosed herein will determine how robust the whole chain of DRs and AMs is to disruption or overexpression of a single gene involved in the chain. Perturbation of some Type B genes may affect such a chain.

Among other advantages, the method disclosed herein facilitates splitting a set of differentially expressed genes into two distinct categories to be assessed separately. The method disclosed herein also suggests a radical conceptual change in current approaches focused solely on differentially expressed genes.

In an embodiment, any of the analytical methods and processes described above, as desired, are implemented using a computer system. For example, the process described with reference to the flow chart of FIG. 1 can be automated using a computer. As a further example, any of the algorithms and evaluative processes described elsewhere in this specification can be implemented using a computer system.

A more rigorous test of the hypothesis that a gene pair is a Type A pair will now be presented. Testing the hypothesis H₀: the gene pair under consideration is a Type A pair is equivalent to testing the hypothesis: ξ and ζ=ψ−ξ are stochastically independent. Testing independence is more cumbersome and time-consuming than testing the absence of correlation and this is why we confine ourselves to the latter approach. However, our sample analyses have shown that the results of the two tests are in good agreement. Presented below is a statistical test for the hypothesis H₀⁽¹⁾: ρ(ξ,ζ)=0, where ρ(ξ,ζ) is the correlation coefficient between ξ and ζ=ψ−ξ. A test statistic for the hypothesis H₀⁽¹⁾ is given by the Fisher's transformation

$$ρ ( ξ , ζ ) = \frac{1}{2} log \frac{1 + ω ( ξ , ζ )}{1 - ω ( ξ , ζ )} ,$$

where ω is the Pearson sample correlation coefficient. Under the null hypothesis, the statistic ρ has an asymptotic normal distribution with mean 0 and standard deviation 1/\sqrt{n−3}.

However, the situation is more complicated if there is a multiplicative array-specific technological noise in the data because the correlation structure of the vector (ξ,ζ) becomes non-identifiable. The most widely accepted noise model assumes that the same multiplicative measurement error is shared by all genes on each array, but the level of this error varies randomly from array to array. In the presence of this random effect denoted by A, we observe (ξ+α,ψ+α), where α=log A and A is a positive r.v. (random variable) independent of the pair (Ξ, Ψ). Now we deal with the pair of r.v.s (ξ+α,ζ), where ζ=(ψ+α)−(ξ+α). It is interesting that the hypothesis of independence for ξ+α and ζ is equivalent to the hypothesis of independence for ξ and ζ. For simplicity we formulate this result in terms of the original r.v.'s A, Ξ,Ψ and Z; it obviously remains valid for their logarithms as well.

Proposition. Let (Ξ,Ψ) be a random vector with non-negative components and let A be a positive r.v. independent of (Ξ,Ψ). Suppose that there exists δ>0 such that EX^{s}<δ, EΨ^{σ}<δ, EA^{s}<δ for every |σ|<δ. The r.v.'s AΞ and Z=Ψ/Ξ are independent if and only if Ξ and Z are independent.

Proof. From the well-known properties of the Mellin transform, it follows that the r.v.'s Ξ and Z are independent if and only if

E(X^{s}Z^{t})=E(X^{s}) E(Z^{t})

for all |σ|, |τ|<™. Since A is independent of the pair (Ξ,Ψ), we can write

E{(AX)^{s}Z^{t}}=E{A^{s}Y^{t}X^{s-t}}=E(A^{s}) E{X^{s}Z^{t}}.

Then the following implications are obvious:

E{AX)^{s}Z^{t}}=E(A^{s}X^{s})E(Z^{t}) [symbol] E(A^{s})E(X^{s}Z^{t})=E(A^{s})E(X^{s})E(Z^{t}) [symbol] E(X^{s}Z^{t})=E(X^{s})E(Z^{t}),

given E(A^{s})>0. This proves our proposition.

Because of this nice property, the test statistic given by the Fisher's transformation can be applied to noisy data in order to test the hypothesis H₀⁽¹⁾. However, the noise adds variability to the data and this may affect the error properties of the test. The latter effect is impossible to assess even by simulations because the noise is unobservable. Since our main goal is to demonstrate the extent of the phenomenon under study by conservatively estimating the abundance of Type A pairs, we are much more concerned with Type 2rather than Type 1 errors. With this in mind, we reformulate the problem of hypothesis testing as follows.

Let the random vector u=(u₁, . . . , uₘ) represent logarithms of the true expression levels of m genes and let a=(a, . . . , a) be the corresponding m-dimensional noise vector with identical components. The observed expression levels are represented as v=u+a. It is assumed that all the r.v.'s under consideration have finite second moment. Let us choose two components of the vector u and denote them by x and y, respectively. The corresponding components of the vector v are given by
ξ=x+a and η=y+a,
where the r.v. α is unobservable, of course. For definiteness sake we always assume that σ²(ξ)≦σ²(η). Introduce the class [symbol] = [symbol] (u′) of all random μ-dimensional vectors u′ such that u′+a′=v, where a=(a′. . . , a′) and a′is a random variable (on the log-scale) independent of u′. For the pair (ξ,η) we test the hypothesis

$${H}_{0}^{( 2 )} \text{:} {sup}_{{u}^{′} ∈} \vert ρ ( {ξ}^{′} , {η}^{′} - {ξ}^{′} ) \vert = 0 ,$$

where ξ′ and η′ are any two components of the vector u′. It is easy to show that

$$ρ ( {ξ}^{′} , {η}^{′} - {ξ}^{′} ) = ρ ( ξ , η - ξ ) {\{ 1 + \frac{{σ}^{2} ( {a}^{′} )}{{σ}^{2} ( {ξ}^{′} )} \}}^{\frac{1}{2}} ,$$

where ξ′ and η′ are independent. We know that σ²(a)≦σ²(a)+σ²(uₖ) for any component ω̅_{κ}of the vector v, κ=1, . . . , μ. Therefore,

$${σ}^{2} ( a ) \leq {σ}^{2} = {min}_{1 \leq k \leq m} {σ}^{2} ( {v}_{k} ) , \qquad ( 8 )$$

where σ²(vₖ) is the variance of observed (noisy) expression of the κth gene and the minimum in (8) is taken over all the genes. Notice also that σ²(Uₖ)≧σ²(vₖ)−σ² for any κ=1, . . . , μ. Since σ²(ξ′)≧σ², the following inequality holds:

$${sup}_{{u}^{′} ∈} \vert {ρ}^{2} ( {ξ}^{′} , {η}^{′} - {ξ}^{′} ) \vert \leq \vert ρ ( ξ , η - ξ ) \vert {\{ 1 + \frac{{σ}_{2}}{{σ}^{2} ( ξ ) - {σ}^{2}} \}}^{\frac{1}{2}} , \qquad ( 9 )$$

where all the characteristics involved can be estimated for all genes except the one for which the minimum in (8) is attained. The latter gene is excluded from the analysis.

By replacing all the parameters in the right-hand side of inequality (9) with their empirical estimators and transforming the resultant expression in accordance with the Fisher's transformation, we obtain a test-statistic for rejecting H₀⁽²⁾. Let us denote this statistic by ρ*. The upper bound in (9) is sharp as it can be shown that the bound is attained if all components of the vector v are normally distributed. In the latter case, the test based on ρ* controls a given nominal significance level for testing the hypothesis H₀⁽²⁾. Otherwise, the test thus designed may have a higher actual significance level than a given nominal level, which is of little concern when estimating the abundance of Type A pairs because we make the estimate more conservative by falsely rejecting more true null hypotheses. However, this circumstance should be kept in mind when using the test to select individual Type B pairs in combination with multiple testing procedures. Another important caveat is that the above test cannot be used for selecting individual Type A pairs. As is the case with the statistic ρ given by the Fisher's transformation, the asymptotic distribution of ρ* is normal but with a larger variance. Therefore, we make our test only more rejective (at a sacrifice in the Type 1 error rate) when constructing ρ* from the noisy data but using quantiles of the sampling distribution for the statistic ρ.

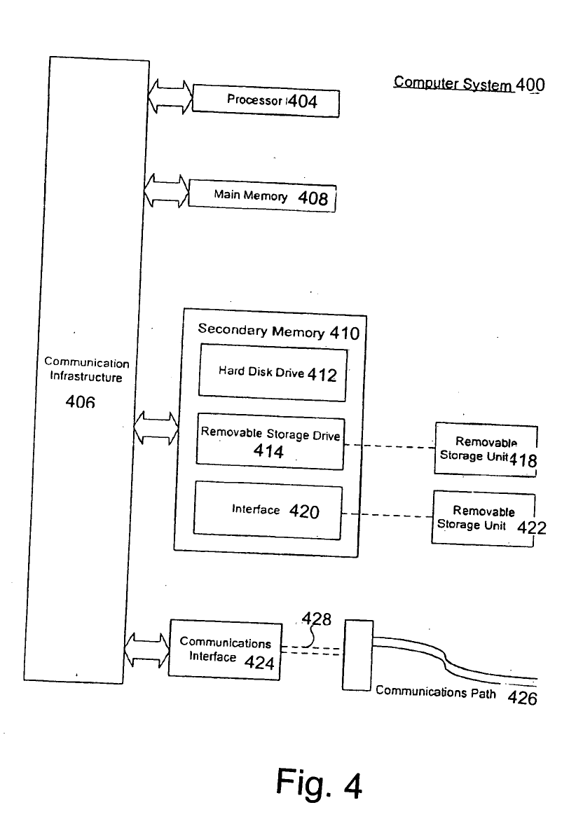

The following description of a general purpose computer system, such as a PC system, is provided as a non-limiting example of systems on which the disclosed analysis can be performed. In particular, the methods disclosed herein can be performed manually, implemented in hardware, or implemented as a combination of software and hardware. Consequently, desired features of the invention may be implemented in the environment of a computer system or other processing system. An example of such a computer system 400 is shown in FIG. 4. The computer system 400 includes one or more processors, such as processor 404. Processor 404 can be a special purpose or a general purpose digital signal processor. The processor 404 is connected to a communication infrastructure 406 (for example, a bus or network). Various software implementations are described in terms of this exemplary computer system. After reading this description, it will become apparent to a person skilled in the relevant art how to implement the invention using other computer systems and/or computer architectures.

Computer system 400 also includes a main memory 405, preferably random access memory (RAM), and may also include a secondary memory 410. The secondary memory 410 may include, for example, a hard disk drive 412, and/or a RAID array 416, and/or a removable storage drive 414, representing a floppy disk drive, a magnetic tape drive, an optical disk drive, etc. The removable storage drive 414 reads from and/or writes to a removable storage unit 418 in a well known manner. Removable storage unit 418, represents a floppy disk, magnetic tape, optical disk, etc. As will be appreciated, the removable storage unit 418 includes a computer usable storage medium having stored therein computer software and/or data.

In alternative implementations, secondary memory 410 may include other similar means for allowing computer programs or other instructions to be loaded into computer system 400. Such means may include, for example, a removable storage unit 422 and an interface 420. Examples of such means may include a program cartridge and cartridge interface (such as that found in video game devices), a removable memory chip (such as an EPROM, or PROM) and associated socket, and other removable storage units 422 and interfaces 420 which allow software and data to be transferred from the removable storage unit 422 to computer system 400.

Computer system 400 may also include a communications interface 424. Communications interface 424 allows software and data to be transferred between computer system 400 and external devices. Examples of communications interface 424 may include a modem, a network interface (such as an Ethernet card), a communications port, a PCMCIA slot and card, etc. Software and data transferred via communications interface 424 are in the form of signals 428 which may be electronic, electromagnetic, optical or other signals capable of being received by communications interface 424. These signals 428 are provided to communications interface 424 via a communications path 426. Communications path 426 carries signals 428 and may be implemented using wire or cable, fiber optics, a phone line, a cellular phone link, an RF link and other communications channels.

The terms “computer program medium” and “computer usable medium” are used herein to generally refer to media such as removable storage drive 414, a hard disk installed in hard disk drive 412, and signals 428. These computer program products are means for providing software to computer system 400.

Computer programs (also called computer control logic) are stored in main memory 408 and/or secondary memory 410. Computer programs may also be received via communications interface 424. Such computer programs, when executed, enable the computer system 400 to implement the present invention as discussed herein. In particular, the computer programs, when executed, enable the processor 404 to implement the processes of the present invention. Where the invention is implemented using software, the software may be stored in a computer program product and loaded into computer system 400 using raid array 416, removable storage drive 414, hard drive 412 or communications interface 424.

The present invention has been described above with the aid of functional building blocks and method steps illustrating the performance of specified functions and relationships thereof. The boundaries of these functional building blocks and method steps have been arbitrarily defined herein for the convenience of the description. Alternate boundaries can be defined so long as the specified functions and relationships thereof are appropriately performed. Any such alternate boundaries are thus within the scope and spirit of the claimed invention. One skilled in the art will recognize that these functional building blocks can be implemented by discrete components, application specific integrated circuits, processors executing appropriate software and the like or any combination thereof. Thus, the breadth and scope of the present invention should not be limited by any of the above-described exemplary embodiments, but should be defined only in accordance with the claims and their equivalents.

While various embodiments of the present invention have been described above, it should be understood that they have been presented by way of example only, and not limitation. For example, numerical values are illustrative rather than limiting, as are specific mathematical techniques. Thus, the breadth and scope of the present invention should not be limited by any of the above-described exemplary embodiments, but should be defined only in accordance with the following claims and their equivalents.

We claim:

1. A method for establishing investigative priorities for genes, comprising:
a) determining values for a variable X representing an expression level of a first gene;
b) determining values for a variable Y representing an expression level of a second gene;
c) defining the first gene and second gene as having a first relationship type if X and Y satisfy the condition Y=X Z (where Z≧1) and X and Z are estimated to be substantially stochastically independent variables, and as having a second relationship type if the first and second gene do not have the first relationship type; and
d) generating an output of results identifying one or more genes having the second relationship type.

2. The method of claim 1, further comprising (e) using the output in establishing investigative priorities for gene evaluation.

3. The method of claim 1, wherein step (c) comprises determining whether X and Z are substantially non-correlated random variables and identifying X and Z as stochastically independent based on non-correlation.

4. The method of claim 1, comprising the further step of repeating steps (a), (b), and (c) iteratively for each driver gene identified as having the first relationship type to identify another gene that is a driver for the identified driver gene.

5. The method of claim 1, wherein step (c) comprises determining whether x and z=y -x are stochastically independent, where x=log X, y=log Y. and z=log Z.

6. A device for establishing investigative priorities for genes, comprising:
an input for receiving values for a variable X representing an expression level of a first gene and values for a variable Y representing an expression level of a second gene;
a processor, in communication with the input, for defining the first gene and second gene as having a first relationship type if X and Y satisfy the condition Y=X Z (where Z≧1) and X and Z are estimated to be substantially stochastically independent variables, and as having a second relationship type if the first and second gene do not have the first relationship type, and for generating an output of results identifying one or more genes having the second relationship type; and
an output, in communication with the processor, for outputting the output of results generated by the processor.

7. The device of claim 6, wherein the processor determines whether X and Z are substantially non-correlated random variables and identifying X and Z as stochastically independent based on non-correlation.

8. The device of claim 6, wherein the processor operates iteratively for each driver gene identified as having the first relationship type to identify another gene that is a driver for the identified driver gene.

9. The device of claim 6, wherein step (c) comprises determining whether x and z=y−x are stochastically independent, where x=log X, y=log Y, and z=log Z.

10. An article of manufacture for establishing investigative priorities for genes, comprising:
a computer-readable storage medium; and
code, stored on the computer-readable storage medium, for:
a) determining values for a variable X representing an expression level of a first gene;
b) determining values for a variable Y representing an expression level of a second gene;
c) defining the first gene and second gene as having a first relationship type if X and Y satisfy the condition Y=X Z (where Z≧1) and X and Z are estimated to be substantially stochastically independent variables, and as having a second relationship type if the first and second gene do not have the first relationship type; and
d) generating an output of results identifying one or more genes having the second relationship type.
